(19) United States
(12) Patent Application Publication
(10) Pub. No.: US 2024/0389923 A1
(43) Pub. Date: Nov. 28, 2024

(54) MUSCLE RELAXATION MONITORING DEVICE AND NON-TRANSITORY COMPUTER READABLE STORAGE MEDIUM

(71) Applicant: NIHON KOHDEN CORPORATION, Tokyo (JP)

(72) Inventor: Hiroshi YOSHIHARA, Tokorozawa-shi (JP)

(73) Assignee: NIHON KOHDEN CORPORATION, Tokyo (JP)

(21) Appl. No.: 18/662,571

(22) Filed: May 13, 2024

(30) Foreign Application Priority Data
May 22, 2023 (JP) 2023-084124

(51) Int. Cl. A61B 5/395

(52) U.S. Cl. CPC A61B 5/395

(57) ABSTRACT

A muscle relaxation monitoring device includes a stimulation output unit configured to stimulate a nerve via a stimulation electrode attached to a living body of a subject, a signal detection unit configured to detect, as an electrical signal, a physiological signal generated from a muscle in response to the stimulation to the nerve by the stimulation output unit, via a lead-out electrode attached to the living body, and a controller configured to cause the stimulation output unit to stimulate the nerve, and determine an abnormal installation cause of the lead-out electrode, based on the electrical signal detected by the signal detection unit.

### CROSS-REFERENCE TO RELATED APPLICATIONS

This application is based on and claims priority under 35 USC 119 from Japanese Patent Application No. 2023-084124 filed on May 22, 2023, the entire content of which is incorporated herein by reference.

### TECHNICAL FIELD

The presently disclosed subject matter relates to a muscle relaxation monitoring device and a non-transitory computer readable storage device.

### BACKGROUND ART

Although anesthesia is generally used when surgery is performed on a patient, a muscle may contract due to reflection or the like even when the patient is not conscious or perceived due to the anesthesia. Therefore, in large surgery, a muscle relaxant is administered to a patient, and the surgery is facilitated by preventing the patient from moving. A surgery preparation such as administration of an anesthesia drug is performed before the start of the surgery, and a muscle relaxant is also administered at this time. After the muscle relaxant is administered, a muscle relaxation state is monitored in order to confirm whether the patient is in the muscle relaxation state. In addition, an effect of the muscle relaxant decreases with time, and thus the muscle relaxation state is monitored and the muscle relaxant is re-administered in accordance with a progress of surgery. As a device configured to monitor a muscle relaxation state, a muscle relaxation monitoring device configured to stimulate a nerve of a patient and to measure contraction of a muscle caused by the stimulation is used, as described in JP2006-326050A and JP2021-69724A. JP2006-326050A discloses a muscle relaxation monitoring device configured to measure, with an acceleration degree, motion caused by muscular contraction due to nerve stimulation.

On the other hand, JP2021-69724A discloses an electromyogram-base muscle relaxation monitoring device configured to electrically stimulate a nerve and to measure a physiological signal generated from a muscle as a reaction thereof. In the muscle relaxation monitoring device, two stimulation electrodes are attached in a vicinity of the nerve, and two lead-out electrodes are attached in a vicinity of the muscle that contracts due to the nerve stimulation. The nerve is stimulated by causing a stimulation current to flow from a stimulation output unit through the stimulation electrodes, and a physiological signal generated from the muscle at that time is detected by a signal detection unit as an electrical signal obtained through the lead-out electrodes.

In the electromyogram-base muscle relaxation monitoring device, the stimulation current flows, an electromyogram that is a biological reaction is obtained, and a muscle relaxation state is monitored. As a basic way of use, before the muscle relaxant is administered, a current value of the stimulation current is set, and the current value of the stimulation current is adjusted.

In the electromyogram-base muscle relaxation monitoring device, abnormal installation such as peeling of the electrode or failure of an attachment position may occur in a surgery preparation stage or during surgery. There are various abnormal installation causes of the muscle relaxation monitoring device, and when there is an abnormality, it is necessary to consider all possibilities and respond accordingly. Therefore, it may take time to prepare the muscle relaxation monitoring device and recover from an abnormal installation.

### SUMMARY OF INVENTION

Aspect of non-limiting embodiments of the present disclosure relates to make it possible to identify a cause for an abnormality of a muscle relaxation monitoring device at the time of surgery preparation or the like, thereby quickly responding to the abnormality and quickly performing the surgery preparation or the like.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a muscle relaxation monitoring device including:
a stimulation output unit configured to stimulate a nerve via a stimulation electrode attached to a living body of a subject;
a signal detection unit configured to detect, as an electrical signal, a physiological signal generated from a muscle in response to the stimulation to the nerve by the stimulation output unit, via a lead-out electrode attached to the living body; and
a controller configured to:
   cause the stimulation output unit to stimulate the nerve; and
   determine an abnormal installation cause of the lead-out electrode, based on the electrical signal detected by the signal detection unit.

According to an aspect of the present disclosure, there is provided a non-transitory computer readable storage medium storing a muscle relaxation monitoring program including instructions which, when executed by a computer, cause the computer to perform:
a stimulation procedure of stimulating a nerve via a stimulation electrode attached to a living body, by a stimulation output unit,
a detection procedure of detecting, as an electrical signal, a physiological signal generated from a muscle in response to the stimulation to the nerve by the stimulation output unit, via a lead-out electrode attached to the living body, by a signal detection unit, and
a determination procedure of:
   causing the stimulation output unit to stimulate the nerve; and
   determining an abnormal installation cause, based on the electrical signal detected by the signal detection unit.

### BRIEF DESCRIPTION OF DRAWINGS

Exemplary embodiment(s) of the present invention will be described in detail based on the following figures, wherein:

FIG. 1 illustrates a configuration of a muscle relaxation monitoring system including a muscle relaxation monitoring device according to an exemplary embodiment of the present invention;

FIG. 2 is an electromyogram of a living body to which no muscle relaxant is administered according to the exemplary embodiment of the present invention;

FIG. 3 is an electromyogram, obtained by a TOF method, of the living body to which no muscle relaxant is administered according to the exemplary embodiment of the present invention;

FIG. 4 is an electromyogram obtained by the TOF method when the muscle relaxant is not completely acting according to the exemplary embodiment of the present invention;

FIG. 5 illustrates a first abnormal waveform according to the exemplary embodiment of the present invention;

FIG. 6 illustrates a second abnormal waveform according to the exemplary embodiment of the present invention;

FIG. 7 illustrates a third abnormal waveform according to the exemplary embodiment of the present invention; and

FIG. 8 is a flowchart for determining and displaying an abnormal installation cause at the time of setting a current value of a stimulation current according to the exemplary embodiment of the present invention.

### DESCRIPTION OF EMBODIMENTS

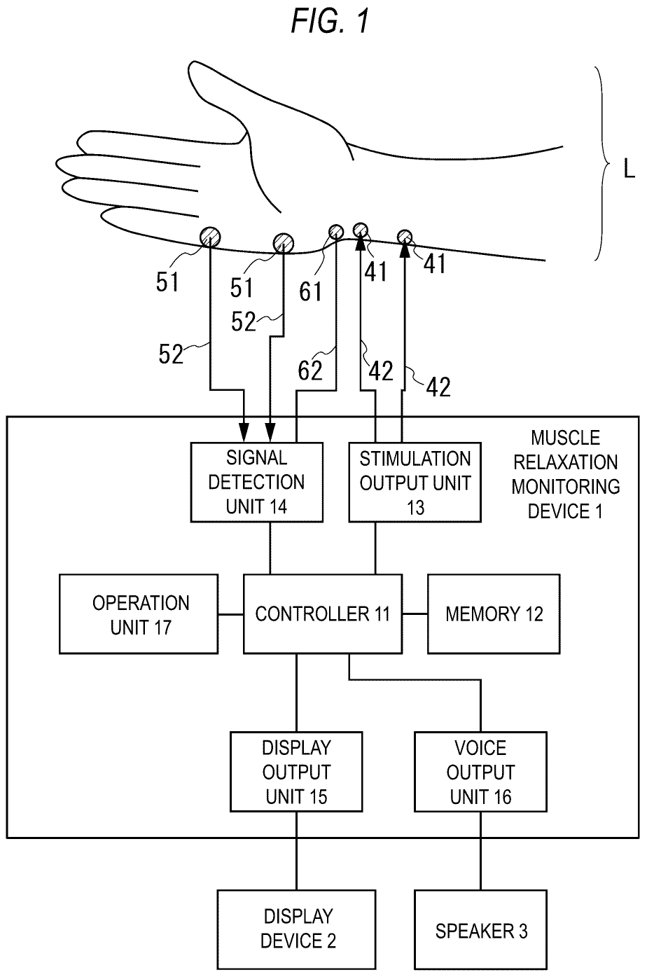

FIG. 1 illustrates a configuration of a muscle relaxation monitoring system, according to an embodiment, attached to an upper limb L of a subject who is a patient. The muscle relaxation monitoring system can include a muscle relaxation monitoring device 1, a display device 2, a speaker 3, stimulation electrodes 41, wires 42, lead-out electrodes 51, signal lines 52, a ground electrode 61, and a ground line 62. A pair of stimulation electrodes 41 and a pair of wires 42 are provided, and a pair of lead-out electrodes 51 and a pair of signal lines 52 are also provided. The display device 2 and the speaker 3 may be incorporated in the muscle relaxation monitoring device 1.

The muscle relaxation monitoring device 1 can include a controller 11, a memory 12, a stimulation output unit 13, a signal detection unit 14, a display output unit 15, a voice output unit 16, and an operation unit 17. The controller 11 includes a CPU. The display output unit 15 and the voice output unit 16 correspond to a notification output unit for notifying of a muscle relaxation monitoring result, and also function as a notification output unit for notifying of an abnormality of a stimulation current, an abnormal installation cause, a response content according to the abnormal installation cause, and the like.

The memory 12, the stimulation output unit 13, the signal detection unit 14, the display output unit 15, the voice output unit 16, and the operation unit 17 are connected to the controller 11. The stimulation output unit 13 is connected to the pair of stimulation electrodes 41 via the pair of wires 42. The signal detection unit 14 is connected to the pair of lead-out electrodes 51 via the pair of signal lines 52. Further, the signal detection unit 14 is connected to the ground electrode 61 via the ground line 62. The stimulation electrodes 41, the lead-out electrodes 51, and the ground electrode 61 are surface electrodes attached to a human body. The display output unit 15 of the muscle relaxation monitoring device 1 is connected to the display device 2. The voice output unit 16 is connected to the speaker 3.

In the upper limb L of the subject illustrated in FIG. 1, an ulnar nerve extends inside an arm. The ulnar nerve is a nerve that causes an adductor pollicis muscle, an abductor digiti minimi muscle, and the like in a hand to contract. In a case where the muscle relaxation monitoring device 1 is used, the stimulation electrodes 41, the lead-out electrodes 51, and the ground electrode 61 are attached to a living body. The pair of stimulation electrodes 41 are attached to positions corresponding to the ulnar nerve. The pair of lead-out electrodes 51 are attached to positions corresponding to the abductor digiti minimi muscle and a tendon in a vicinity of the abductor digiti minimi muscle. The ground electrode 61 is attached between the stimulation electrodes 41 and the lead-out electrodes 51.

The signal detection unit 14 is configured to detect a potential difference between two physiological signals, input via the signal lines 52, generated in the abductor digiti minimi muscle and in the tendon. The pair of stimulation electrodes 41 are attached along the ulnar nerve, and the stimulation output unit 13 is configured to cause a pulsed stimulation current to flow through the wires 42 and the stimulation electrodes 41 and to stimulate the ulnar nerve. In the present embodiment, a period during which the stimulation current flows is 0.2 msec. Then, the signal detection unit 14 is configured to detect, as an electrical signal between the pair of lead-out electrodes 51, a physiological signal generated from a muscle in response to the stimulation to the ulnar nerve. The signal detection unit 14 includes a differential amplifier and is configured to amplify a potential difference between the pair of lead-out electrodes 51. Then, the signal detection unit 14 is configured to perform A/D conversion on the amplified voltage value and to send the converted voltage value to the controller 11. The ground electrode 61 is configured to prevent a noise voltage transmitted from the body of the subject to the upper limb L from affecting a detection potential that passes through the lead-out electrodes 51. The noise voltage is mainly caused by a voltage pulse caused by the stimulation current or by propagation of the voltage pulse caused by the stimulation current in the body of the subject.

Derived data detected and A/D converted by the signal detection unit 14 is input to the controller 11, and a muscle relaxation state is monitored. A monitoring result is displayed on the display device 2 via the display output unit 15, and a sound is notified of from the speaker 3 via the voice output unit 16.

The muscle relaxation monitoring device 1 is configured to adjust a stimulation current value or the like by operating a switch on the operation unit 17. The controller 11 and the memory 12 also function as a stimulation current value setting unit configured to adjust the stimulation current in a surgery preparation stage to set a stimulation current value. Further, the controller 11 and the memory 12 also function as a cause determination unit configured to determine an abnormal installation cause of the lead-out electrodes 51 or the like, based on the electrical signal detected by the signal detection unit 14.

As described above, in the muscle relaxation monitoring system, as illustrated in FIG. 1, the muscle relaxation monitoring device 1 is connected to the upper limb L of the subject, and the stimulation current flows from the stimulation output unit 13. Then, the signal detection unit 14 is configured to detect, as an electrical signal to obtain an electromyogram, the physiological signal generated from the muscle. The stimulation current from the stimulation output unit 13 flows between the two stimulation electrodes 41 and stimulates the ulnar nerve. The physiological signal generated from the muscle connected to the ulnar nerve is detected by the signal detection unit 14 as an electrical signal of a voltage between the two lead-out electrodes 51. The physiological signal changes depending on the muscle relaxation state caused by an action of a muscle relaxant.

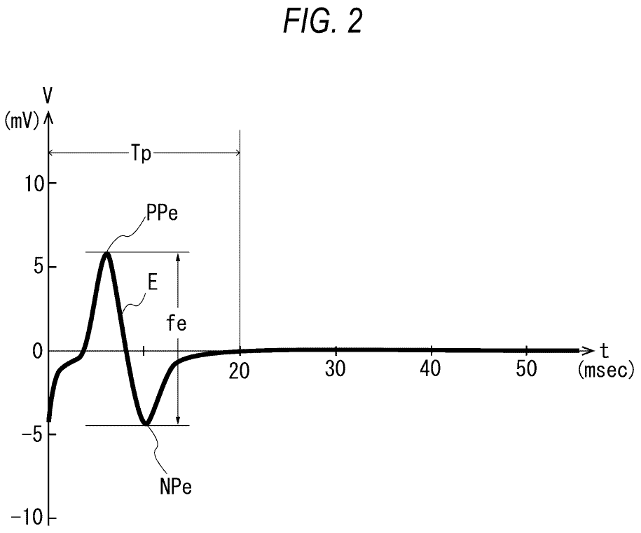

FIG. 2 illustrates an example of an electromyogram of the living body to which no muscle relaxant is administered. The electromyogram shows a potential difference between electrical signals obtained from the two lead-out electrodes 51 attached to the upper limb L of the subject. A vertical axis represents a potential difference, and a horizontal axis represents a time. In order to avoid direct noise due to the stimulation current from appearing in the drawing, the electromyogram in FIG. 2 shows the potential difference between the two lead-out electrodes 51 immediately after a pulse of a stimulation current of 0.2 msec ends. The electromyogram in FIG. 2 shows a non-administration waveform E of the living body to which no muscle relaxant is administered.

The non-administration waveform E illustrated in FIG. 2 starts from a negative potential of a residual voltage due to the stimulation current, becomes a positive peak potential PPe of 6 mV at about 6 msec, becomes a negative peak potential NPe of −4 mV at about 11 msec, and then converges to a potential of 0 mV at about 20 msec. In FIG. 2, a physiological signal period Tp, which is a period during which a physiological signal is generated from the muscle due to the stimulation current to the nerve, is 0 msec to about 20 msec. The non-administration waveform E generally has a shape as illustrated in FIG. 2, although a voltage value of a peak potential, a timing, and the like slightly differ depending on an attachment state of electrodes, characteristics of the living body, and the like.

On the other hand, in a case where a muscle relaxant is administered, no physiological signal is generated at a maximum muscle relaxation degree, a potential of the electrical signal becomes 0, and no positive peak potential PPe or negative peak potential NPe is generated. In addition, in a case where the muscle relaxation degree decreases, a physiological signal is generated, but the physiological signal gradually decreases each time a plurality of stimulations are performed in a short time. By using this phenomenon, the muscle relaxation monitoring device 1 of the embodiment can monitor the muscle relaxation degree by using a train of four (TOF) method. The muscle relaxation monitoring device 1 can obtain an electromyogram using not only the TOF method but also various stimulations.

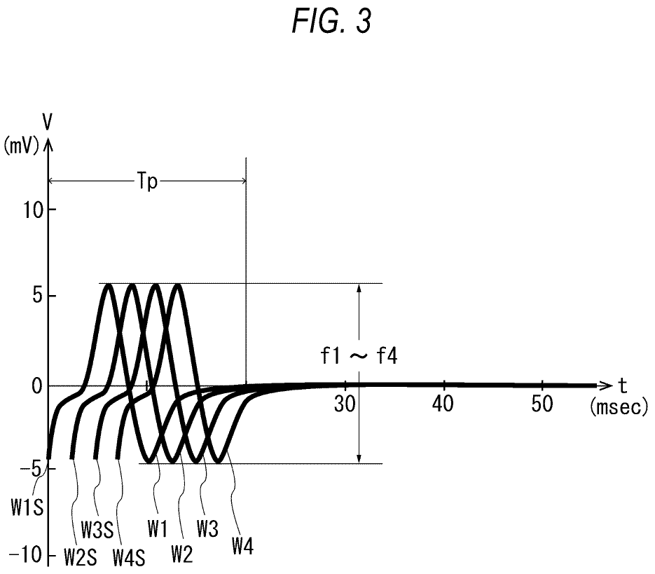

FIG. 3 illustrates an electromyogram representing physiological signals in a case where stimulations are performed by using the TOF method before administration of a muscle relaxant. In the embodiment, the muscle relaxation degree is calculated by using the TOF method. In the TOF method of the embodiment, a stimulation is performed by repeating one group every 15 seconds, with one group consisting of four consecutive stimulations every 500 msec. A period of a stimulation current for stimulation is 0.2 msec. The stimulation is performed by causing a stimulation current to flow from the stimulation output unit 13 between the stimulation electrodes 41 illustrated in FIG. 1. FIG. 3 illustrates four muscle potential waveforms W1 to W4 sequentially generated due to four consecutive stimulations. A vertical axis represents a voltage value V, and a horizontal axis represents a time t.

The physiological signal period Tp, which is a time width in which potentials of the muscle potential waveforms W1 to W4 fluctuate, is shorter than 500 msec, which is a stimulation interval. Therefore, the muscle potential waveforms W1 to W4 in FIG. 3 are displayed with stimulation timings shortened for convenience and start points shifted little by little in a positive direction of the time t. A numerical value of the time t in FIG. 3 indicates a time of the muscle potential waveform W1 corresponding to an initial stimulation. A start point W1S of the muscle potential waveform W1 is 0 msec. A start point W2S of the muscle potential waveform W2 is at a time position of 500 msec in a t direction, and is shifted in a −t direction as illustrated in FIG. 3. The same applies to the muscle potential waveforms W3 and W4, and start points W3S and W4S are at time positions of 1000 msec and 1500 msec, respectively. In order to avoid noise due to the stimulation current, as in FIG. 2, the muscle potential waveforms W1 to W4 indicate potential differences immediately after pulses of stimulation currents end.

Before the administration of the muscle relaxant, the muscle potential waveforms W1 to W4 have shapes of the non-administration waveform E illustrated in FIG. 2, and the physiological signal periods Tp in the muscle potential waveforms W1 to W4 have the same length. FIG. 3 illustrates only the physiological signal period Tp of the muscle potential waveform W1. As illustrated in FIG. 3, amplitudes f1 to f4, which are differences between a highest potential and a lowest potential in the physiological signal period Tp in the muscle potential waveforms W1 to W4, have the same magnitude. Before the administration of the muscle relaxant, the amplitudes of the muscle potential waveforms do not decrease while one group of stimulation currents are applied.

In the TOF method, a ratio (TOF ratio) of the amplitude f1 of the muscle potential waveform W1 due to a first stimulation to the amplitude f4 of the muscle potential waveform W4 due to a fourth stimulation, is expressed in % as an index of the muscle relaxation degree. The amplitudes f1 and f4 are voltage values of the differences between the highest potential and the lowest potential in the physiological signal periods Tp in the muscle potential waveforms W1 and W4, respectively. In a case where the muscle relaxant is not effective at all, the TOF ratio is 100%. In FIG. 3 which is the electromyogram before the administration of the muscle relaxant, f1 =f4, and the TOF ratio is 100%.

In a case where the muscle relaxant is administered and the muscle relaxation degree is maximized, all of the muscle potential waveforms W1 to W4 become 0 mV, although not illustrated. All the amplitudes f1 to f4 are 0 mV. In this state, even in a case where the nerve is stimulated by the stimulation current, no physiological signal is generated in the muscle, and the potential difference between the pair of lead-out electrodes 51 is 0 mV.

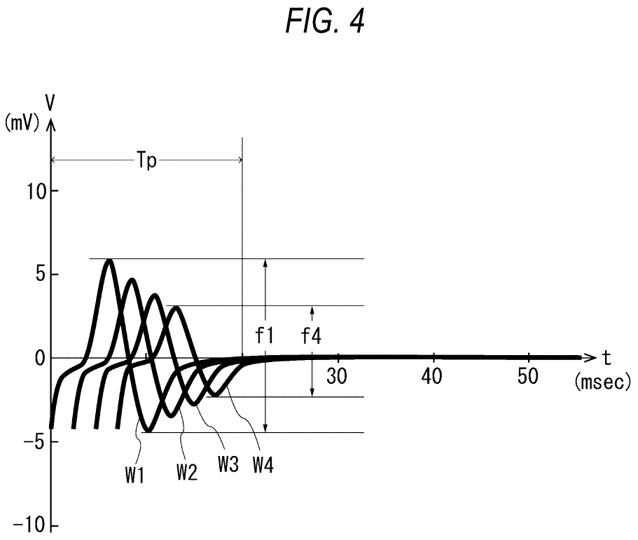

On the other hand, at a muscle relaxation degree at which the action of the muscle relaxant is weak, such as when a time elapses after the muscle relaxant is administered, an amplitude f of the muscle potential waveform decreases as the number of stimulations increases in one group of stimulations. FIG. 4 illustrates an electromyogram representing physiological signals in a case where stimulations are performed by using the TOF method at this time. In FIG. 4, a stimulation is performed by repeating one group every 15 seconds, with one group consisting of four consecutive stimulations every 500 msec. A period of a stimulation current for stimulation is 0.2 msec. The stimulation is performed by causing a stimulation current to flow between the stimulation electrodes 41 illustrated in FIG. 1.

FIG. 4 illustrates the four muscle potential waveforms W1 to W4 sequentially generated due to four consecutive stimulations. A vertical axis represents a voltage value V, and a horizontal axis represents a time t. The physiological signal periods Tp in the muscle potential waveforms W1 to W4 have the same length. FIG. 4 illustrates only the physiological signal period Tp of the muscle potential waveform W1. The physiological signal periods Tp of the muscle potential waveforms W1 to W4 are shorter than 500 msec which is a stimulation interval, and thus stimulation timings are shortened for convenience, and start points are shifted little by little in a positive direction of the time t in FIG. 4. A numerical value of the time t in FIG. 4 indicates a time of the muscle potential waveform W1 corresponding to an initial stimulation. Same or similarly to FIGS. 2 and 3, the muscle potential waveforms W1 to W4 indicate potential differences immediately after pulses of stimulation currents end. In FIG. 4, for the amplitudes f1 to f4 in the muscle potential waveforms W1 to W4, only the amplitudes f1 and f4 are illustrated, and the amplitudes f2 and f3 are not illustrated.

At a muscle relaxation degree at which an action of a muscle relaxant is weak, as illustrated in FIG. 4, the amplitude f1 of the muscle potential waveform W1 is a difference between a highest potential and a lowest potential in the physiological signal period Tp during which the electromyogram is generated. The physiological signal period Tp illustrated in FIG. 4 corresponds to the muscle potential waveform W1. Same or similarly, each of the amplitudes f2 to f4 in the muscle potential waveforms W2 to W4 also indicates a difference between a highest potential and a lowest potential in a period during which the electromyogram is generated. In the muscle potential waveforms W1 to W4 of the muscle relaxation degree at which the action of the muscle relaxant is weak illustrated in FIG. 4, the amplitudes f1 to f4 satisfy f1 >f2 >f3 >f4, and gradually decrease.

At the muscle relaxation degree at which the action of the muscle relaxant illustrated in FIG. 4 is weak, the TOF ratio=f4/f1 is less than 50%. It is considered that a muscle is recovered from the muscle relaxation state when the TOF ratio is >70%, and thus in the state of the electromyogram illustrated in FIG. 4, the muscle is in a process of recovering from the muscle relaxation state and is not recovered from the muscle relaxation state. In FIG. 4, the muscle potential waveform W1 is the same waveform as the non-administration waveform E, but the amplitude f1 of the muscle potential waveform W1 may be smaller than an amplitude fe of the non-administration waveform E. However, even in a case where the amplitude f1 decreases, the amplitude f4 further decreases, and thus the TOF ratio can be used as an index of the muscle relaxation degree.

The TOF ratio is monitored during surgery and is used for determination of the re-administration of the muscle relaxant or the like. For example, in a case where the TOF ratio becomes 40% or more, the re-administration of the muscle relaxant or the like is determined. In addition, in the surgery preparation stage, it is possible to confirm based on the TOF ratio whether the patient is in a muscle relaxation state in which the patient can perform the surgery. When the TOF ratio is >70%, the muscle is recovered from the muscle relaxation state, and in recent years, 80% or 90% may be a standard value.

### Use Form of Muscle Relaxation Monitoring Device 1

Next, a use form of the muscle relaxation monitoring device 1 according to the embodiment will be described. First, in a surgery preparation stage, as illustrated in FIG. 1, the pair of stimulation electrodes 41, the pair of lead-out electrodes 51, and the ground electrode 61 are attached to the upper limb L of the subject. Before a muscle relaxant is administered, a current value of a stimulation current of the muscle relaxation monitoring device 1 is set.

In a case where a stimulation current value setting switch is turned on by the operation unit 17, the muscle relaxation monitoring device 1 sets the current value of the stimulation current. In the present embodiment, an abnormal installation cause is determined in a case where the stimulation current value setting unit sets the current value of the stimulation current. In the setting of the current value of the stimulation current, the current value of the stimulation current is gradually increased and automatically adjusted to an appropriate value. In a case where an electromyogram is measured by using the TOF method with the muscle relaxation monitoring device 1 in which adjustment is completed, as illustrated in FIG. 3, the TOF ratio is 100%.

After the setting of the current value of the stimulation current is completed, a muscle relaxant is administered to the subject. Due to the muscle relaxant, the subject gradually enters a muscle relaxation state. In a complete muscle relaxation state, a voltage of the electromyogram is 0 mV. As illustrated in FIG. 4, the amplitudes f1 to f4 of the muscle potential waveforms W1 to W4 gradually decrease in the electromyogram obtained by the TOF method in the course of the complete muscle relaxation state, after the muscle relaxant is administered. The muscle potential waveforms W1 to W4 are waveforms of the electromyogram sequentially generated due to stimulation currents every 500 msec.

In the course of the complete muscle relaxation state, as illustrated in FIG. 4, due to the action of the muscle relaxant, the amplitudes of the muscle potential waveforms W1 to W4 become smaller as the consecutive stimulations become slower. In FIG. 4, the TOF ratio is smaller than 70%, and thus it can be said that the subject enters the muscle relaxation state. In a case where the action of the muscle relaxant further progresses, the muscle potential waveforms W1 to W4 become substantially 0 mV, and the amplitudes f1 to f4 of the muscle potential waveforms W1 to W4 also become substantially 0 mV, and the subject enters a complete muscle relaxation state. After the muscle relaxation monitoring device 1 confirms the complete muscle relaxation state, surgery is started.

In a case where the action of the muscle relaxant becomes weak after a time elapses from the administration of the muscle relaxant, potentials in the muscle potential waveforms W1 to W4 change as illustrated in FIG. 4 again. Then, in a case where the TOF ratio becomes larger than a certain value, it is determined whether the muscle relaxant is administered again according to a progress state of the surgery or the like.

As described above, in the muscle relaxation monitoring device 1, a stimulation procedure and a detection procedure are performed by using the TOF method. In the stimulation procedure, the stimulation output unit 13 stimulates a nerve via the stimulation electrodes 41 attached to the living body. In the detection procedure, a physiological signal generated from a muscle in response to the stimulation to the nerve by the stimulation output unit 13 is detected by the signal detection unit 14, as an electrical signal, via the lead-out electrodes 51 attached to the living body. The stimulation procedure and the detection procedure are controlled by the controller 11 performing processing based on a program stored in the memory 12.

### Determination of Abnormal Installation Cause

In a case where the stimulation current value setting switch is turned on in the surgery preparation stage, the muscle relaxation monitoring device 1 performs the stimulation procedure and the detection procedure as in the case of the muscle relaxation monitoring. In the stimulation procedure, the stimulation output unit 13 stimulates a nerve via the stimulation electrodes 41 attached to the living body. In the detection procedure, a physiological signal generated from a muscle in response to the stimulation to the nerve by the stimulation output unit 13 is detected by the signal detection unit 14, as an electrical signal, via the lead-out electrodes 51 attached to the living body. The stimulation procedure and the detection procedure are controlled by the controller 11 performing processing based on a program stored in the memory 12.

As a result of performing the stimulation procedure and the detection procedure for setting the stimulation current in the surgery preparation stage, when an abnormal waveform is obtained without obtaining an electromyogram as illustrated in FIG. 2, it is considered that the abnormal installation of the muscle relaxation monitoring device 1 is caused. Same or similarly, when an abnormal waveform is obtained during surgery, it is considered that the abnormal installation of the muscle relaxation monitoring device 1 is caused. The abnormal waveform may have a plurality of shapes. The muscle relaxation monitoring device 1 is configured to perform a determination procedure of stimulating the nerve by the stimulation output unit 13 and of determining an abnormal installation cause based on the electrical signal detected by the signal detection unit 14. The determination procedure is controlled by the controller 11 performing processing based on the program stored in the memory 12.

### First Abnormal Waveform A1

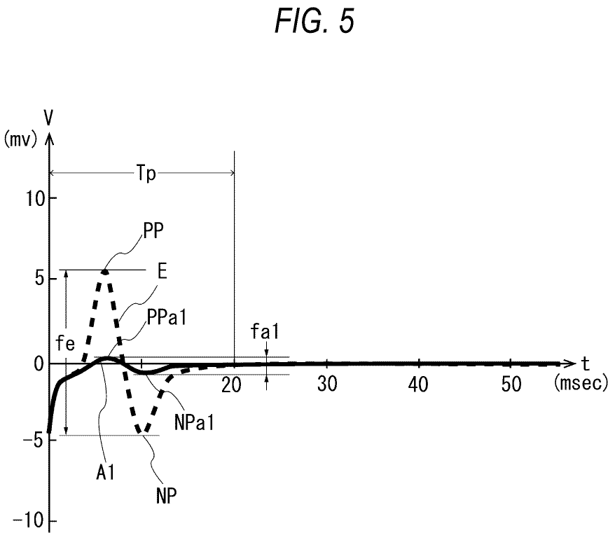

In FIG. 5, an electrical signal of the first abnormal waveform A1 detected by the signal detection unit 14 after the stimulation to the nerve by the stimulation output unit 13 is indicated by a solid line. A dotted line indicates the non-administration waveform E, which is an electromyogram of the living body to which no muscle relaxant is administered illustrated in FIG. 2. The first abnormal waveform A1 has a positive peak potential PPa1 and a negative peak potential NPa1 smaller than those of the non-administration waveform E in the physiological signal period Tp. There is an amplitude fa1, which is a difference between a highest potential and a lowest potential in a period during which an electromyogram is generated, and the amplitude fa1 is smaller than the amplitude fe of the non-administration waveform E.

An electromyogram like the first abnormal waveform A1 is generated when the stimulation to the nerve is insufficient. When the two lead-out electrodes 51 are too close to each other, it is difficult to capture a potential difference due to a physiological signal, resulting in the first abnormal waveform A1. As described above, after the stimulation to the nerve by the stimulation output unit 13, in a case where the difference between the highest potential and the lowest potential in the period during which the electromyogram is generated is in a predetermined voltage range smaller than that in a normal electromyogram, the muscle relaxation monitoring device 1 determines that the electromyogram is the first abnormal waveform A1. The determination of the first abnormal waveform A1 means that there may be two causes, that is, insufficient stimulation to the nerve and abnormal approach of the attachment positions of the two lead-out electrodes 51.

In the muscle relaxation monitoring device 1 of the present embodiment, when the amplitude fa1 which is a difference between the positive peak potential PPa1 and the negative peak potential NPa1 is equal to or larger than 0.2 mV which is a first reference value SV11 and is equal to or smaller than 1.0 mV which is a second reference value SV12, it is determined that the electromyogram is the first abnormal waveform A1. A range equal to or larger than the first reference value SV11 and equal to or smaller than the second reference value SV12 corresponds to the predetermined voltage range that is smaller than that in the normal electromyogram. The second reference value SV12 is set to a value smaller than the amplitude fe of the non-administration waveform E. The second reference value SV12 is preferably a value equal to or smaller than ½ of the amplitude fe of the non-administration waveform E. The first reference value SV11 is set to such a level that the positive peak potential PPa1 and the negative peak potential NPa1 can be recognized.

In the setting of the current value of the stimulation current, the stimulation current flows a plurality of times with the gradually increased current value, but the stimulation current value has an upper limit. Then, it is determined whether the electromyogram corresponding to the stimulation current when the stimulation current value reaches the upper limit, is the first abnormal waveform A1. The determination of the first abnormal waveform A1 based on output of the signal detection unit 14 is a determination that the abnormal installation cause is whether the stimulation to the nerve is insufficient, the attachment positions of the two lead-out electrodes 51 are too close to each other, or the attachment positions are deviated from the tendon or a muscle.

The abnormal installation cause as a determination result is output from the display output unit 15 and displayed on the display device 2. The display device 2 is configured to display “insufficient stimulation to the nerve or abnormal approach of the attachment positions of the lead-out electrodes”. At the time of display, a voice signal is output from the voice output unit 16, and a sound “beep” is output from the speaker 3 to call attention.

The display at the time of the determination may be a display content for notifying of a response content according to the abnormal installation cause. In this case, for example, a message that “Please attach stimulation electrodes securely. In a case where the abnormality is not resolved even in this case, please separate and reattach the lead-out electrodes” is displayed.

In a case where the first abnormal waveform A1 is obtained in a muscle relaxation monitoring state during surgery, the amplitude may have become smaller due to the action of the muscle relaxant. Therefore, even in a case where the first abnormal waveform A1 is obtained, it is not determined that the cause is the insufficient stimulation to the nerve or the like.

### Second Abnormal Waveform A2

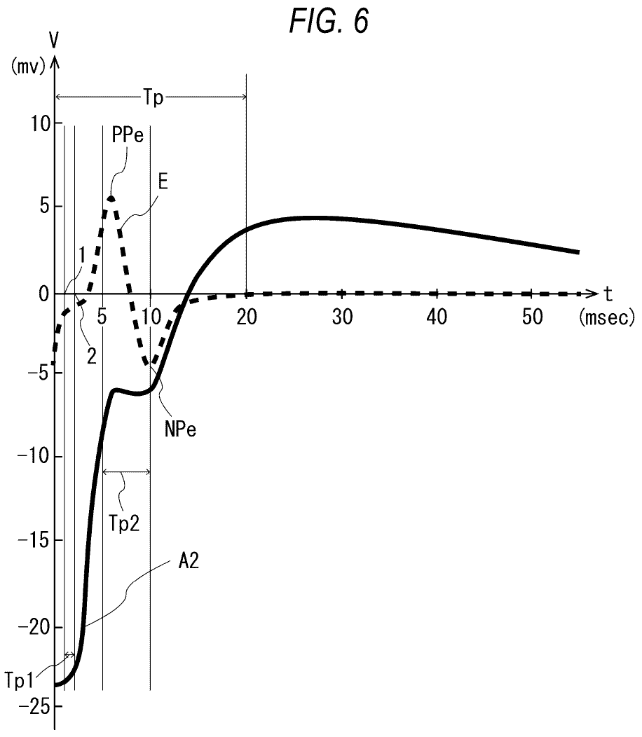

In FIG. 6, an electrical signal of the second abnormal waveform A2 detected by the signal detection unit 14 after the stimulation to the nerve by the stimulation output unit 13, is indicated by a solid line. A dotted line indicates the non-administration waveform E which is an electromyogram of the living body to which no muscle relaxant is administered illustrated in FIG. 2. The second abnormal waveform A2 is a waveform in which the waveform of the non-administration waveform E is distorted, and an absolute value of an initial voltage of the electromyogram is abnormally large. The electromyogram in FIG. 6 also indicates changes in the electromyogram from an end time point of the stimulation current.

It is considered that the electromyogram like the second abnormal waveform A2 indicated by the solid line in FIG. 6 is generated due to an influence of stimulation artifact noise remaining after the end time point of the stimulation current and generated due to the stimulation current. The ground electrode 61 is provided, and thus the influence of the stimulation artifact noise on the electrical signal detected by the signal detection unit 14 can be prevented. However, when the ground electrode 61 is peeled off, the remaining stimulation artifact noise is not prevented, resulting in an electromyogram like the second abnormal waveform A2. In addition, when the stimulation electrode 41 and the lead-out electrode 51 are too close to each other, the influence of the remaining stimulation artifact noise becomes large, resulting in the second abnormal waveform A2.

The stimulation artifact noise greatly affects the electrical signal detected by the signal detection unit 14 during a predetermined period after the end time point of the stimulation current. In a case where the electrical signal during the predetermined period after the stimulation to the nerve by the stimulation output unit 13 is distorted, it is determined that the electromyogram is the second abnormal waveform A2. The determination of the second abnormal waveform A2 means that the abnormal installation cause is an abnormality in which the stimulation electrode 41 and the lead-out electrode 51 are too close to each other or an abnormal peeling of the ground electrode 61.

In the muscle relaxation monitoring device 1 of the present embodiment, as illustrated in FIG. 6, in a case where an absolute value of an average voltage in a first period Tp1 which is an initial period of the electromyogram is 20 mV or more and an absolute value of an average voltage in a second period Tp2 is 10 mV or less, it is determined that the electromyogram is the second abnormal waveform A2. The first period Tp1 is 1 msec to 2 msec from the end time point of the stimulation current, and the second period Tp2 is 5 msec to 10 msec. In this way, by determining the absolute values of the average voltages in the first period Tp1 and the second period Tp2, it is possible to determine the distortion of the electrical signal in the physiological signal period Tp which is the predetermined period. The first period Tp1 and the second period Tp2 can be appropriately set at the time of manufacturing or before the use of the muscle relaxation monitoring device 1.

In the setting of the current value of the stimulation current in the surgery preparation or the like, the stimulation current flows a plurality of times with the gradually increased current value. In this case, it is determined whether an electromyogram corresponding to a stimulation current having a maximum current value is the second abnormal waveform A2.

The determination of the second abnormal waveform A2 based on the output of the signal detection unit 14 is a determination by the controller 11 as to whether the abnormality in which the stimulation electrode 41 and the lead-out electrode 51 are too close to each other or the abnormal peeling of the ground electrode 61 is the abnormal installation cause. The abnormal installation cause as a determination result is output from the display output unit 15 and displayed on the display device 2. The display device 2 is configured to display “the abnormality in which the stimulation electrode and the lead-out electrode are too close to each other or the abnormal peeling of the ground electrode”. At the time of display, a voice signal is output from the voice output unit 16, and a sound “beep” is output from the speaker 3 to call attention.

The display at the time of the determination may be a display content for notifying of a response content according to the abnormal installation cause. In this case, for example, a message that “Please attach the ground electrode securely. In a case where the abnormality is not resolved even in this case, please separate the attachment positions of the stimulation electrode and the lead-out electrode” is displayed.

### Third Abnormal Waveform A3

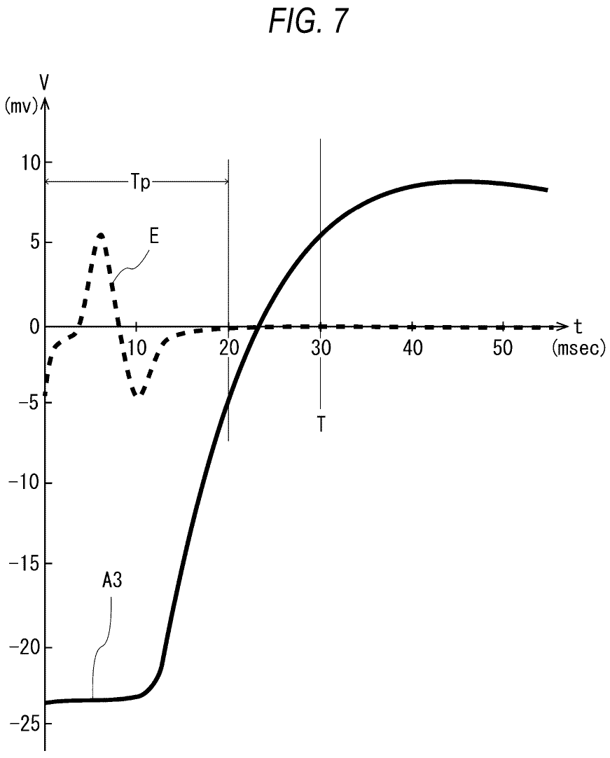

In FIG. 7, an electrical signal of the third abnormal waveform A3 detected by the signal detection unit 14 after the stimulation to the nerve by the stimulation output unit 13 is indicated by a solid line. A dotted line indicates the non-administration waveform E, which is an electromyogram of the living body to which no muscle relaxant is administered illustrated in FIG. 2. The first abnormal waveform A1 and the second abnormal waveform A2 have traces of the positive peak potential PPe and negative peak potential NPe due to the physiological signals, but the third abnormal waveform A3 does not have these traces.

The third abnormal waveform A3 appears when the lead-out electrode 51 is peeled off. When the lead-out electrode 51 is peeled off, a waveform like the third abnormal waveform A3 may be obtained, and a waveform having another shape may also be obtained. However, when the lead-out electrode 51 is peeled off, any waveform is less likely to converge to a potential of 0 mV after the physiological signal period Tp. The non-administration waveform E indicated by the dotted line in FIG. 7 converges to the potential of 0 mV during the physiological signal period Tp. On the other hand, the third abnormal waveform A3 indicated by the solid line has a potential of about 5 mV in 30 msec from the end time point of the stimulation current after the physiological signal period Tp.

As illustrated in FIG. 6, same as or similarly to the third abnormal waveform A3, the second abnormal waveform A2 converges to the potential of 0 mV slowly. Therefore, in the muscle relaxation monitoring device 1 of the present embodiment, when an absolute value of a voltage at a determination time point T after 30 msec from the end time point of the stimulation current after the physiological signal period Tp is equal to or larger than 2 mV which is a predetermined value, and it is not determined that the electromyogram is the second abnormal waveform A2, it is determined that the electromyogram is the third abnormal waveform A3. The determination time point T can be appropriately set at the time of manufacturing or before the use of the muscle relaxation monitoring device 1. As described above, when an absolute value of a potential after the period during which the physiological signal is generated after the stimulation to the nerve by the stimulation output unit 13 is equal to or larger than the predetermined value, it is determined that the abnormal installation cause is an abnormal peeling of the lead-out electrode 51.

In the setting of the current value of the stimulation current in the surgery preparation or the like, the stimulation current flows a plurality of times with the gradually increased current value. In this case, it is determined whether an electromyogram corresponding to a stimulation current having a maximum current value is the third abnormal waveform A3.

The determination of the third abnormal waveform A3 based on the output of the signal detection unit 14 is a determination by the controller 11 that the abnormal peeling of the lead-out electrode 51 is the abnormal installation cause. The abnormal installation cause as a determination result is output from the display output unit 15 and displayed on the display device 2. The display device 2 is configured to display that “There is an abnormal peeling of the lead-out electrode.”. At the time of display, a voice signal is output from the voice output unit 16, and an alerting sound “beep” is output from the speaker 3 to call attention.

The display at the time of the determination may be a display content for notifying of a response content according to the abnormal installation cause. In this case, for example, a message “Please attach the lead-out electrode securely.” is displayed.

### Determination Flow of Abnormal Installation Cause

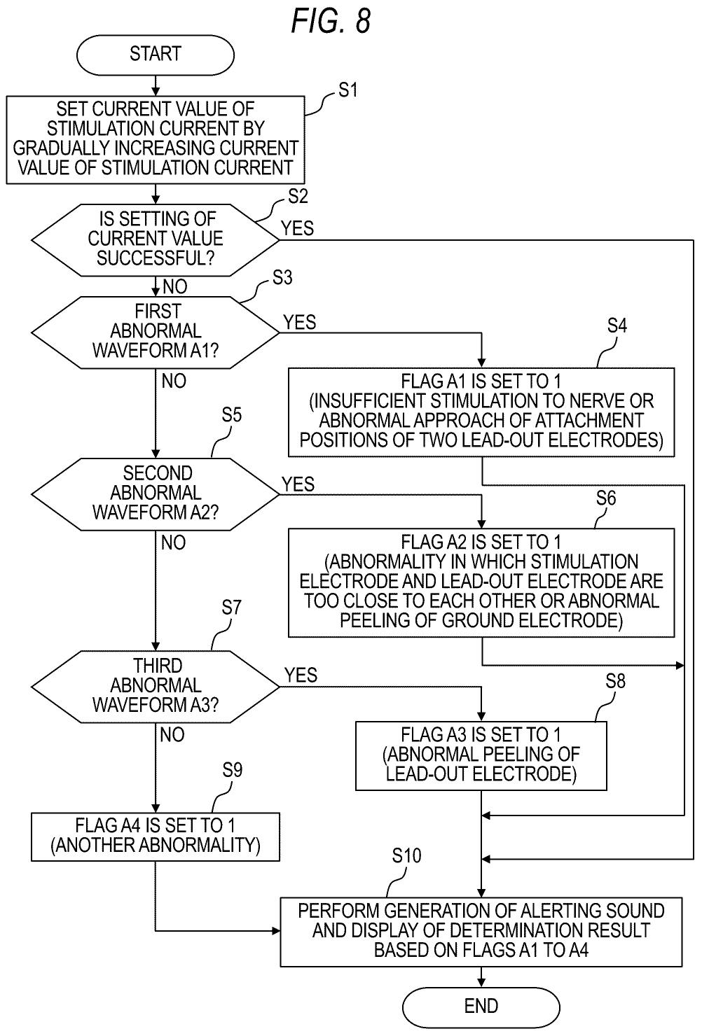

FIG. 8 illustrates a flowchart for determining and displaying the abnormal installation cause. A procedure according to steps in the flowchart shows a muscle relaxation monitoring method. In addition, the procedure is controlled by the controller 11 performing processing based on a muscle relaxation monitoring program stored in the memory 12. The muscle relaxation monitoring program can be recorded in a non-transitory computer readable recording storage medium.

In the present embodiment, an abnormal installation cause is determined in a case where the stimulation current value setting unit sets the current value of the stimulation current. At a start time point, flags A1 to A4 to be described later become 0. In a case where the stimulation current value setting switch (not illustrated) is turned on by the operation unit 17 illustrated in FIG. 1, a stimulation current value setting start signal is sent to the controller 11. The controller 11 sends a stimulation application signal to the stimulation output unit 13, and repeats pulses of a stimulation current having a gradually increased current value to set a current value of the stimulation current (step S1).

As illustrated in FIG. 2, in a case where an electromyogram having the positive peak potential PPe and the negative peak potential NPe in an assumed voltage range during an assumed physiological signal period Tp is obtained, the setting of the current value of the stimulation current is successful. In a case where it is determined that the setting of the current value of the stimulation current is successful (YES in step S2), the current value of the stimulation current at that time is stored, and the process proceeds to step S10, where a display indicating the completion of the setting of the current value of the stimulation current is displayed. The current value when the setting of the current value of the stimulation current is successful is stored as a current setting value, and is used as a current value of a stimulation current in the TOF method.

On the other hand, in a case where the current value of the stimulation current is gradually increased and no electromyogram having the positive peak potential PPe and the negative peak potential NPe in the assumed voltage range during the assumed physiological signal period Tp, as illustrated in FIG. 2, at the time of the maximum current value is obtained, and NO is determined in step S2, an electromyogram corresponding to the stimulation current having the maximum current value is stored.

Then, it is determined whether the stored electromyogram is the first abnormal waveform A1 illustrated in FIG. 5 (step S3). In a case where the amplitude fa1, which is the difference between the positive peak potential PPa1 and the negative peak potential NPa1 during the physiological signal period Tp in the electromyogram, is equal to or larger than 0.2 mV which is the first reference value SV11 and is equal to or smaller than 1.0 mV which is the second reference value SV12, it is determined that the electromyogram is the first abnormal waveform A1. When it is determined that the electromyogram is the first abnormal waveform A1 (YES in step S3), the flag A1 is set to 1 (step S4). The flag A1 of “1” means insufficient stimulation to the nerve or abnormal approach of the attachment positions of the two lead-out electrodes 51. In a case where a difference between a highest potential and a lowest potential in a period during which a physiological signal is generated after the stimulation to the nerve by the stimulation output unit 13 is in the predetermined voltage range smaller than that in the normal electromyogram, it is determined that the abnormal installation cause is the insufficient stimulation to the nerve or the abnormal approach of the attachment positions of the two lead-out electrodes 51. As described above, it is determined that the abnormal installation cause is the insufficient stimulation to the nerve or the abnormal approach of the attachment positions of the two lead-out electrodes 51.

When it is determined that the electromyogram is not the first abnormal waveform A1 (NO in step S3), it is determined whether the stored electromyogram is the second abnormal waveform A2 illustrated in FIG. 6 (step S5). In a case where the absolute value of the average voltage in the first period Tp1 of 1 msec to 2 msec from the end time point of the stimulation current is 20 mV or more and the absolute value of the average voltage in the second period Tp2 of 5 msec to 10 msec from the end time point of the stimulation current is 10 mV or less, it is determined that the electromyogram is the second abnormal waveform A2. When it is determined that the electromyogram is the second abnormal waveform A2 (YES in step S3), the flag A2 is set to 1 (step S6). The flag A2 of “1” means the abnormality in which the stimulation electrode 41 and the lead-out electrode 51 are too close to each other or the abnormal peeling of the ground electrode 61. As described above, it is determined whether the abnormal installation cause is the abnormality in which the stimulation electrode 41 and the lead-out electrode 51 are too close to each other or the abnormal peeling of the ground electrode 61.

When it is determined that the electromyogram is not the second abnormal waveform A2 (NO in step S5), it is determined whether the stored electromyogram is the third abnormal waveform A3 illustrated in FIG. 7 (step S7). In a case where it is determined that the absolute value of the voltage at the determination time point T after 30 msec from the end time point of the stimulation current is 2 mV or more, it is determined that the electromyogram is the third abnormal waveform A3. When it is determined that the electromyogram is the third abnormal waveform A3 (YES in step S5), the flag A3 is set to 1 (step S6). The flag A3 of “1” means the abnormal peeling of the lead-out electrode 51.

In the flow, step S7 of determining whether the electromyogram is the third abnormal waveform A3 is a determination performed when it is determined that the electromyogram is not the second abnormal waveform A2, and thus it is not necessary to determine in step S7 that the electromyogram is not the second abnormal waveform A2. As described above, it is determined whether the abnormal installation cause is the abnormality in which the stimulation electrode 41 and the lead-out electrode 51 are too close to each other or the abnormal peeling of the ground electrode 61.

Setting the flags A1 to A3 to 1 corresponds to determining that the electromyograms are the first abnormal waveform A1 to the third abnormal waveform A3. The determination of the first abnormal waveform A1 means that the abnormal installation cause is the insufficient stimulation to the nerve or the abnormal approach of the attachment positions of the two lead-out electrodes 51. The determination of the second abnormal waveform A2 means that the abnormal installation cause is the abnormality in which the stimulation electrode 41 and the lead-out electrode 51 are too close to each other or the abnormal peeling of the ground electrode 61. The determination of the third abnormal waveform A3 means that the abnormal installation cause is the abnormal peeling of the lead-out electrode 51. When it is determined that the electromyogram is not the third abnormal waveform A3 (NO in step S7), the flag A4 is set to 1 (step S9). The flag A4 of “1” means that there is another abnormality.

After steps S4, S6, S8, and S9, the generation of the alerting sound and the display of the determination result are further performed based on the flags A1 to A4 (step S10). When any one of the flags A1 to A4 is 1, the controller 11 causes the voice output unit 16 to output a sound and causes the speaker 3 to generate an alerting sound “beep”. When all the flags A1 to A4 are 0, a sound “pip” is generated. In addition, the controller 11 causes the display output unit 15 to display and output the determination result on the display device 2.

When any one of the flags A1 to A4 is 1, the abnormal installation cause corresponding to each flag is displayed. When the flag A1 is 1, a message “the insufficient stimulation to the nerve or the abnormal approach of the attachment positions of the lead-out electrodes” is displayed. In addition, when the flag A2 is 1, a message “the abnormality in which the stimulation electrode and the lead-out electrode are too close to each other or the abnormal peeling of the ground electrode” is displayed. When the flag A3 is 1, a message “the abnormal peeling of the lead-out electrode” is displayed. When the flag A4 is 1, simply a message “abnormal installation” is displayed. In addition, when all of the flags A1 to A4 are 0, a message that “the setting of the current value of the stimulation current is completed” is displayed.

When the abnormal installation is notified of, the stimulation current value setting switch is pressed again after the electrode is reattached. Then, according to the flowchart illustrated in FIG. 8, the setting of the current value of the stimulation current and the determination and display of the abnormal installation cause are performed again. The flowchart is repeated until the setting of the current value of the stimulation current is successfully completed.

The above procedure is mainly the abnormality determination at the time of setting of the current value of the stimulation current during the surgery preparation, and the same abnormality determination may be performed when the muscle relaxation state is monitored by using the TOF method after the setting of the current value of the stimulation current is completed. In this case, the determination is made based on the electromyogram corresponding to the current value of the stimulation current by using the TOF method. In the abnormality determination during surgery, it is not determined in step S3 whether the electromyogram is the first abnormal waveform A1. This is because the electromyogram may have a waveform similar to that of the first abnormal waveform A1 in a state where the muscle relaxant acts.

It is less necessary to determine whether the electromyogram is the first abnormal waveform A1 during surgery. The phrase “after the setting of the current value of the stimulation current is completed” means that the abnormal installation cause is determined once, and thus the abnormal approach of the attachment positions of the two lead-out electrodes 51 is less likely to occur. In addition, it is considered that the insufficient stimulation to the nerve due to the displacement of the stimulation electrodes 41 is less likely to occur. The peeling of the stimulation electrode 41 can be detected by monitoring the stimulation current.

Both a notification of the abnormal installation cause and a notification of the response content may be displayed, or one of them may be displayed. In addition, the notification of the abnormal installation cause and the notification of the response content may be made by a sound from the speaker 3. Both the notification of the abnormal installation cause or a display notification of the response content made by the display device 2 and a sound notification of the response content made by the speaker 3 may be used, or one of them may be used. In this case, the display output unit 15 and the voice output unit 16 function as a notification output unit for notifying of the abnormal installation cause or the response content according to the abnormal installation cause. In addition, as the notification output unit, a light emitter or the like separately provided in the muscle relaxation monitoring device 1 may be turned on or a vibration device provided in the muscle relaxation monitoring device 1 may be vibrated.

The muscle relaxation monitoring device 1 of the embodiment is separated from the display device 2 and the speaker 3. However, these may be integrated into a medical device. In addition, a medical device system in which the muscle relaxation monitoring device 1 is incorporated together with another medical device may be used, and information from the muscle relaxation monitoring device 1 may be notified of using a display device, a speaker, or the like of the other medical device.

In the embodiment, the use of the muscle relaxation monitoring device 1 before the administration of the muscle relaxant during the surgery preparation has been mainly described. However, the muscle relaxation monitoring device 1 can also be used when any of the first abnormal waveform A1 to the third abnormal waveform A3 is generated, for example, when the action of the muscle relaxant is weakened during surgery.

In the embodiment, the stimulation electrodes 41 and the lead-out electrodes 51 are connected to the muscle relaxation monitoring device 1 via the wires 42 and the signal lines 52. However, the stimulation electrodes 41 may be connected to a stimulation device, and the stimulation device and the muscle relaxation monitoring device may be connected wirelessly. The lead-out electrodes 51 may be connected to a signal detection device, and the signal detection device and the muscle relaxation monitoring device may be connected wirelessly. The wires during surgery can be reduced by the wireless connection.

In addition, the foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A muscle relaxation monitoring device comprising:
a stimulation output unit configured to stimulate a nerve via a stimulation electrode attached to a living body of a subject;
a signal detection unit configured to detect, as an electrical signal, a physiological signal generated from a muscle in response to the stimulation to the nerve by the stimulation output unit, via a lead-out electrode attached to the living body; and
a controller configured to:
cause the stimulation output unit to stimulate the nerve; and
determine an abnormal installation cause of the lead-out electrode, based on the electrical signal detected by the signal detection unit.

2. The muscle relaxation monitoring device according to claim 1, further comprising:
a notification output unit configured to notify of the abnormal installation cause.

3. The muscle relaxation monitoring device according to claim 1, further comprising:
a notification output unit configured to notify of a response content corresponding to the abnormal installation cause.

4. The muscle relaxation monitoring device according to claim 1,
wherein the controller is further configured to set a current value of a stimulation current, and
in a case where the controller sets the current value of the stimulation current, the controller determines the abnormal installation cause.

5. The muscle relaxation monitoring device according to claim 1,
wherein, after the stimulation to the nerve by the stimulation output unit, in a case where a difference between a highest potential and a lowest potential in a period during which a physiological signal is generated is in a predetermined voltage range smaller than that of a normal electromyogram, the controller determines that the abnormal installation cause is an insufficient stimulation to the nerve or is abnormal approach of attachment positions of the two lead-out electrodes.

6. The muscle relaxation monitoring device according to claim 1,
wherein in a case where the electrical signal in a predetermined period after the stimulation to the nerve by the stimulation output unit is distorted, is the controller determines that the abnormal installation cause is an abnormality in which the stimulation electrode and the lead-out electrode are too close to each other or is an abnormal peeling of a ground electrode.

7. The muscle relaxation monitoring device according to claim 1,
wherein, after the stimulation to the nerve by the stimulation output unit, in a case where an absolute value of a potential after a period during which a physiological signal is generated is equal to or larger than a predetermined value, is the controller determines that the abnormal installation cause is an abnormal peeling of the lead-out electrode.

8. A non-transitory computer readable storage medium storing a muscle relaxation monitoring program comprising instructions which, when executed by a computer, cause the computer to perform:
a stimulation procedure of stimulating a nerve via a stimulation electrode attached to a living body, by a stimulation output unit,
a detection procedure of detecting, as an electrical signal, a physiological signal generated from a muscle in response to the stimulation to the nerve by the stimulation output unit, via a lead-out electrode attached to the living body, by a signal detection unit, and
a determination procedure of:
causing the stimulation output unit to stimulate the nerve; and
determining an abnormal installation cause, based on the electrical signal detected by the signal detection unit.
